(19) United States
(12) Patent Application Publication
(10) Pub. No.: US 2026/0354930 A1
(43) Pub. Date: Nov. 12, 2026

(54) Assemblies for Releasably Mounting a Surgical Device to a Patient or Tool

(71) Applicant: Vivid Surgical Pty Ltd, Mosman (AU)

(72) Inventors: William Lindsay Walter, Mosman (AU); Daniel Marsden-Jones, Mosman (AU); Stuart May, Mosman (AU)

(73) Assignee: Vivid Surgical Pty Ltd, Mosman (AU)

(21) Appl. No.: 19/131,940

(22) PCT Filed: Nov. 22, 2023
(86) PCT No.: PCT/AU2023/051192

§ 371 Date: May 21, 2025

(30) Foreign Application Priority Data
Nov. 22, 2022 (AU) 2022903533

(51) Int. Cl. A61B 90/57; A61B 34/20

(52) U.S. Cl. CPC A61B 90/57; A61B 34/20; A61B 2034/2051

(57) ABSTRACT

Assembly for releasably mounting a surgical device to a patient or surgical tool. The assembly includes: a patient mount fixedly securable to a pin configured for engaging the patient, the patient mount defining an operatively top portion carrying at least one first magnetic body; and a device mount fixedly securable to the device and defining an operatively bottom portion carrying a complementary at least one second magnetic body, and defining a recess dimensioned to receive a length of at least one of the pin and the patient mount and allow rotating the device mount relative to the patient mount. Mounting the recess of the device mount on at least one of the pin and the patient mount arranges the bottom portion adjacent and spaced from the top portion. Aligning the at least one first magnetic body with the at least one second magnetic body draws the mounts towards each other to inhibit relative rotation.

### TECHNICAL FIELD

The present disclosure relates, generally, to assemblies for releasably mounting a surgical device to a patient or surgical tool, and, particularly, to such assemblies for mounting a surgical device to a bone pin or a tool shaft.

### BACKGROUND

During surgical procedures, it may be necessary to releasably mount various devices to a patient, such as mounting a digital sensing device to the patient to measure orientation and/or position of the patient, or part of the patient, such as a limb. Other patient-mountable devices include active or passive trackers. Trackers are configurable to allow monitoring orientation and/or position of the patient during surgery by optical imaging, or before or during surgery by radiographic imaging. Such devices may require rigid mounting relative to the patient to inhibit relative movement. This is commonly achieved by mounting the device to one or more bone pins having a threaded end fixed into the patient's bone.

Surgical procedures may also require releasably mounting various devices to a surgical tool, such as mounting a digital sensing device to the tool to measure orientation and/or position of the tool. Such devices may measure orientation and/or position relative to the patient to aid guiding the surgical procedure to enhance accuracy and/or efficiency. These devices may require rigid mounting relative to the tool to inhibit relative movement. This is commonly achieved by mounting the device to the tool with a clamp-type mechanism configured to grip part of the tool, typically being a shaft or handle of the tool.

Some surgical procedures may require moving a device from being mounted to the patient, to being mounted to the tool. In some situations, the device may then be moved back from the tool to the patient. Such scenarios typically require a device mount system which is readily operated by surgical staff and/or causes minimal damage or discomfort to the patient.

Any discussion of documents, acts, materials, devices, articles or the like which has been included in the present specification is not to be taken as an admission that any or all of these matters were common general knowledge in the field relevant to the present disclosure as it existed before the priority date of each of the appended claims.

### SUMMARY

According to some disclosed aspects, there is provided an assembly for releasably mounting a surgical device to a patient. The assembly includes: a patient mount fixedly securable to a pin, the pin configured for threadedly engaging the patient, the patient mount defining an operatively top portion carrying at least one first magnetic body; and a device mount fixedly securable to the surgical device, the device mount defining an operatively bottom portion carrying a complementary at least one second magnetic body, the bottom portion defining a recess dimensioned to receive a defined length of at least one of the pin and the patient mount and allow rotating the device mount relative to the patient mount. One or more of the at least one first magnetic body and the at least one second magnetic body comprises a magnet. Mounting the recess of the device mount on at least one of the pin and the patient mount arranges the bottom portion of the device mount immediately adjacent and spaced from the top portion of the patient mount, and aligning the at least one first magnetic body with the at least one second magnetic body allows the magnets to draw the mounts towards each other to inhibit relative rotation of the mounts.

The patient mount may carry a pair of the first magnetic bodies spaced apart from each other, each of the first magnetic bodies comprising magnets arranged to have corresponding polarity, and the device mount carry a pair of the second magnetic bodies spaced apart from each other, each of the second magnetic bodies comprising magnets arranged to have corresponding polarity to each other and opposite polarity to the first magnetic bodies.

The top portion of the patient mount may carry a third magnetic body interposed between the pair of first magnetic bodies. The third magnetic body may comprise a magnet arranged to have non-corresponding polarity to the first magnetic bodies.

The patient mount may carry a pair of the first magnetic bodies spaced apart from each other, each of the first magnetic bodies comprising permanent magnets, and the device mount carry a pair of the second magnetic bodies spaced apart from each other, each of the second magnetic bodies comprising magnetisable material to allow forming temporary magnets.

The patient mount may be configured to arrange the pair of the first magnetic bodies to be evenly spaced radially away from the pin, and the device mount may be configured to arrange the pair of the second magnetic bodies in complementary spacing to the first magnetic bodies, to allow mounting the device mount on the at least one of the pin and the patient mount and aligning the first magnetic bodies with the second magnetic bodies in one of two relative positions.

The patient mount may be securable along the pin such that an end of the pin is spaced from the top side by more than the defined length to allow mounting the device mount on the pin.

The patient mount may be permanently joined to the pin. The patient mount may be integrally formed with the pin.

According to some disclosed aspects, there is provided an assembly for releasably mounting a surgical device to a patient, the assembly including: a patient mount including a pin configured for threadedly engaging the patient, the patient mount defining an operatively top portion carrying at least one first magnetic body; and a device mount fixedly securable to the surgical device, the device mount defining an operatively bottom portion carrying a complementary at least one second magnetic body, the bottom portion defining a recess dimensioned to receive the patient mount and allow rotating the device mount relative to the patient mount. One or more of the at least one first magnetic body and the at least one second magnetic body comprises a magnet. Mounting the recess of the device mount on the patient mount arranges the bottom portion of the device mount immediately adjacent and spaced from the top portion of the patient mount, and aligning the at least one first magnetic body with the at least one second magnetic body allows the one or more magnets to draw the mounts towards each other to inhibit relative rotation of the mounts.

According to further disclosed aspects, there is provided a surgical device assembly including a surgical navigation device operable to determine orientation, and an assembly as described in any of the preceding paragraphs, where the device mount forms a base portion of the surgical navigation device.

The surgical navigation device may include one or more sensors operable to detect magnetic field, and the surgical navigation device be configured to determine when the device is mounted to the patient mount responsive to the one or more sensors detecting the magnetic field of one or more of the magnetic bodies.

The surgical navigation device may be configured to determine orientation of the device relative to the patient mount responsive to the one or more sensors detecting the magnetic field of one or more of the magnetic bodies.

According to other disclosed aspects, there is provided an assembly for releasably mounting a surgical device to a surgical tool having a shaft, the assembly including: a tool mount having one side defining a receiving portion for receiving part of the shaft, and an opposed side defining an engagement portion, the receiving portion associated with a retention mechanism including a strap securable across the receiving portion, the retention mechanism operable to adjust an effective length of the strap to trap the shaft between the strap and the receiving portion; and a device mount fixedly securable to the surgical device, the device mount configured to releasably engage the engagement portion to allow mounting the surgical device to the surgical tool.

The receiving portion may include at least one pair of abutment surfaces, the surfaces of the, or each, pair arranged to diverge away from each other to allow receiving the part of the shaft between the surfaces.

The receiving portion may include a pair of the abutment surfaces, each of the surfaces being planar.

The tool mount may define an aperture dimensioned to receive the strap, and the retention mechanism be configured to retain the strap in position when arranged to extend through the aperture.

The retention mechanism may include teeth disposed within the aperture, and the strap may be configured to engage the teeth.

The aperture may define a channel housing the teeth, and the strap may define projections shaped to slidingly engage the teeth as the strap is slid into the channel.

The retention mechanism may include a depressible tab associated with the aperture, and the tab be biased towards one side of the aperture to trap the strap and inhibit the strap being drawn through the aperture in at least one direction.

The retention mechanism may include a pivotable member associated with the aperture, and pivoting the pivotable member to a locking position traps the strap to inhibit the strap being drawn through the aperture in at least one direction.

The pivotable member may be arranged such that pivoting the pivotable member towards the engagement portion moves the pivotable member out of the locking position.

The retention mechanism may include hook and loop fasteners fixed relative to the receiving portion, and carried by the strap, and wherein the strap is arrangeable such that the hook and loop fasteners engage to retain the strap in position.

The strap may define a free end arranged to be manually gripped and drawn away from the receiving portion to adjust the effective length.

The strap may define a free end which may define a ring structure shaped to aid gripping.

The strap may define a fixed end releasably secured relative to the receiving portion.

The tool mount and the device mount may each define a portion of a bayonet coupling such that axially engaging and rotating the portions relative to each other engages the mounts together.

The engagement portion may have an operatively top portion defining a protrusion, the top portion carrying at least one first magnetic body, and the device mount may define an operatively bottom portion carrying a complementary at least one second magnetic body, and defining a recess dimensioned to receive at least a portion of the protrusion, and one or more of the at least one first magnetic body and the at least one second magnetic body comprise a magnet, so that mounting the device mount on the protrusion and aligning the at least one first magnetic body with the at least one second magnetic body allows the one or more magnets to draw the mounts towards each other.

The protrusion and recess may be shaped to allow rotation of the tool mount relative to the device mount, so that aligning the at least one first magnetic body with the at least one second magnetic body inhibits relative rotation.

The tool mount may carry a pair of the first magnetic bodies, each of the first magnetic bodies comprising magnets arranged to have corresponding polarity to each other, and the device mount may carry a pair of the second magnetic bodies, each of the second magnetic bodies comprising magnets arranged to have corresponding polarity to each other and opposite polarity to the first magnetic bodies.

The tool mount may carry a pair of the first magnetic bodies, each of the first magnetic bodies comprising magnets arranged to have corresponding polarity, and the device mount carry a pair of the second magnetic bodies, each of the second magnetic bodies formed from ferrous metal.

According to further disclosed aspects, there is provided a surgical device assembly including a surgical navigation device operable to determine orientation, and an assembly as described in any of the preceding paragraphs, where the device mount forms a base portion of the surgical navigation device.

The surgical navigation device may include one or more sensors operable to detect magnetic field and arranged to detect polarity of the at least one second magnetic body, and the surgical navigation device may be configured to determine when the device is mounted to the tool mount responsive to the one or more sensors detecting the magnetic field of the at least one second magnetic body.

In other disclosed aspects, there is provided a kit for releasably mounting a surgical device to a patient or a surgical tool having a shaft, the kit including: a patient mount fixedly securable to a pin, the pin configured for threadedly engaging the patient, the patient mount defining an operatively top portion carrying at least one first magnetic body; a tool mount having one side defining a receiving portion for receiving part of the shaft of the surgical tool, and an opposed side defining an engagement portion, the receiving portion associated with a retention mechanism including a strap securable across the receiving portion, the retention mechanism operable to adjust an effective length of the strap to trap the shaft between the strap and the receiving portion, the engagement portion having an operatively top portion defining a protrusion, and the top portion carrying at least one second magnetic body; and a device mount fixedly securable to the surgical device, the device mount defining an operatively bottom portion carrying at least one third magnetic body, the bottom portion configured to receive part of at least one of the pin and the patient mount, and the bottom portion further configured to at least partially receive the protrusion. At least two of the first, second and third magnetic bodies comprise magnets. Mounting the device mount on at least one of the pin and the patient mount, or on the protrusion, and aligning the at least one third magnetic body with the at least one first magnetic body or the at least one second magnetic body, allows the magnets to draw the mounts towards each other to retain the surgical device to the patient or the surgical tool.

In further disclosed aspects, there is provided a surgical device assembly including a surgical navigation device operable to determine orientation, and an assembly as described in the preceding paragraph, where the device mount forms a base portion of the surgical navigation device.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be appreciated embodiments may comprise steps, features and/or integers disclosed herein or indicated in the specification of this application individually or collectively, and any and all combinations of two or more of said steps or features.

### BRIEF DESCRIPTION OF DRAWINGS

Embodiments will now be described by way of example only with reference to the accompany drawings in which:

FIG. 1 is a front view of an assembly for releasably mounting a surgical device to a patient;

FIG. 2 is a perspective, exploded view of the assembly shown in FIG. 1;

FIG. 3 is a detailed, cross-section front view of the assembly shown in the previous figures;

FIGS. 4A to 4C are top, schematic views of the assembly shown in the previous figures;

FIGS. 5 and 6 are perspective views of a first embodiment of an assembly for releasably mounting a surgical device to a surgical tool having a shaft;

FIGS. 7 and 8 are perspective and cross-section views, respectively, of a second embodiment of an assembly for releasably mounting a surgical device to a surgical tool having a shaft;

FIGS. 9 to 11 are detailed, cross-section front views of a kit for releasably mounting a surgical device to a patient or a surgical tool;

FIGS. 12 to 14 are perspective and cross-section views, respectively, of a third embodiment of an assembly for releasably mounting a surgical device to a surgical tool having a shaft; and

FIGS. 15 and 16 are perspective and cross-section views, respectively, of a third embodiment of an assembly for releasably mounting a surgical device to a surgical tool having a shaft.

### DESCRIPTION OF EMBODIMENTS

In the drawings, reference numeral 10 generally designates an assembly 10 for releasably mounting a surgical device 12 to a patient (not shown). The assembly 10 includes: a patient mount 14 fixedly securable to a pin 16 which is configured for threadedly engaging the patient, the patient mount 14 defining an operatively top portion 18 carrying at least one first magnetic body 20; and a device mount 22 fixedly securable to the device 12, the device mount 22 defining an operatively bottom portion 24 carrying a complementary at least one second magnetic body 26, and the bottom portion 24 defining a recess 28 dimensioned to receive a defined length of at least one of the pin 16 and the patient mount 14 and allow rotating the device mount 22 relative to the patient mount 14. One or more of the at least one first magnetic body 20 and the at least one second magnetic body 26 comprise a magnet. Mounting the recess 28 of the device mount 22 on at least one of the pin 16 and the patient mount 14 arranges the bottom portion 24 of the device mount 22 immediately adjacent and spaced from the top portion 18 of the patient mount 14. Aligning the at least one first magnetic body 20 with the at least one second magnetic body 26 allows the magnet(s) to draw the mounts 14, 22 towards each other to inhibit relative rotation of the mounts 14, 22.

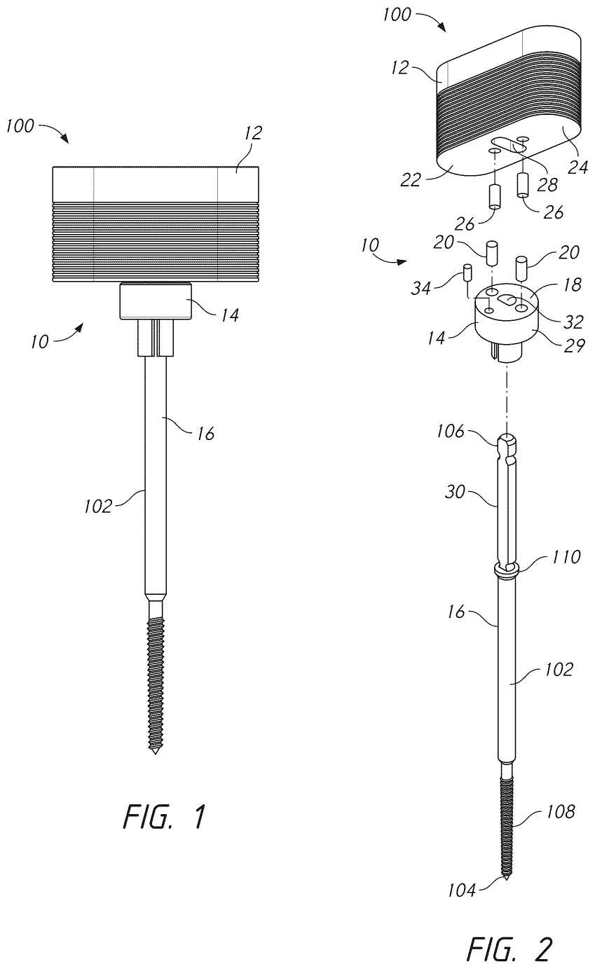

FIGS. 1 to 3 show an embodiment 100 of the assembly 10. In this embodiment 100, the pin 16 is a bespoke bone pin 102 configured for the assembly 100. The bone pin 102 defines opposed ends 104, 106, one end 104 having a thread 108 for threadedly engaging a bone of a patient. A shoulder 110 is defined along the pin 102 to be proximal to and spaced from the other end 106. It will be appreciated that the shoulder 110 is an exemplary structure suitable to support the patient mount 14 and, in other embodiments, this structure is alternatively configurable, such as including any of a recess, indent, rib, spline, or knurled portion.

In this embodiment 100, the patient mount 14 includes a body 29 configured to mounted to the pin 102 at the shoulder 110. The body 29 is slid along the pin 102 to abut the shoulder 110, and then firmly secured to the pin 102 to inhibit relative axial and rotational movement, such as by portion of the body 29 resiliently deforming to clip to the shoulder 110, and/or bonding with adhesive, and/or by operating a fastener, such as a grub screw, where the assembly of the body 29 to the pin 102 is typically performed during factory production. In some embodiments, the body 29 is configured to press-fit to the pin 102.

In other embodiments (not illustrated), the body 29 is overmolded to the pin 102 to effectively integrally form the patient mount 14 with the pin 102. In yet other embodiments (not illustrated), the body 29 is integrally formed with the pin 16, such as defining a radially extending flange of the pin 102. In further embodiments of the assembly 10 (not illustrated), the pin 16 is a conventional bone pin, which typically does not define the shoulder 110, and the patient mount 14 may be manually assembled in the field, such by surgical staff securing the mount 14 to the pin 16. This may involve the body 29 comprising two portions configured to be fitted around the pin 102 and secured together, such as with a clip mechanism and/or fasteners, and/or may involve bonding the patient mount 14 to the pin 16 with adhesive.

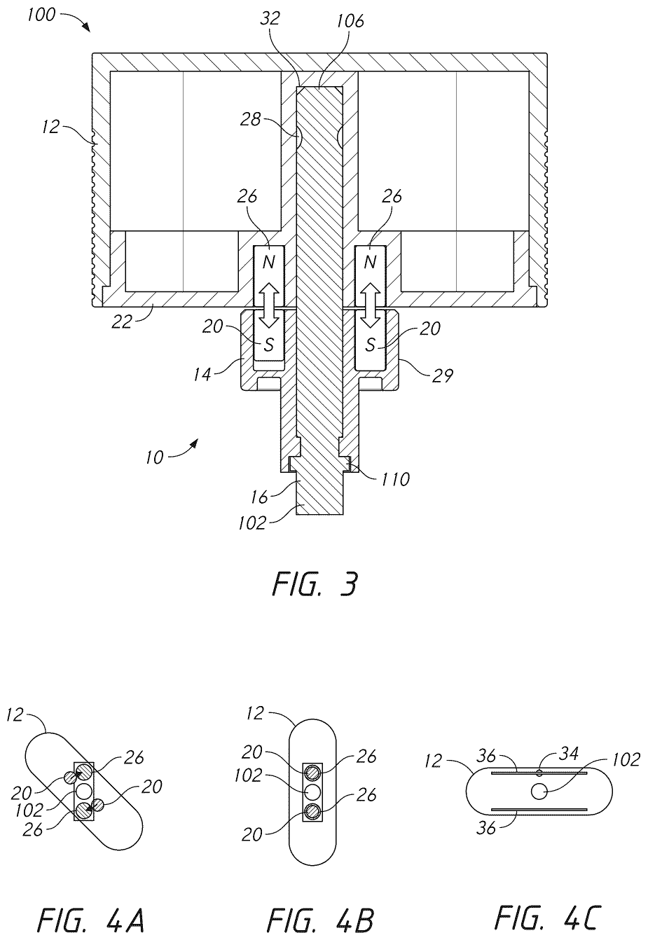

Best shown in FIG. 3, the body 29 of the patient mount 14 is secured along the pin 102 such that the pin 102 extends through the patient mount 14 to arrange the end 106 of the pin 102 to be spaced from the top portion 18 of the mount 14. The spacing between the shoulder 110 and the end 106 arranges the patient mount 14 at a defined distance along the pin 102 away from the end 106, such that a defined portion of the pin 102 protrudes from the mount 14. The protruding portion of the pin 102 forms a mounting axle configured to carry the device mount 22.

In other embodiments (not illustrated), the patient mount 14 is configured to be secured across and receive the end 106 of the pin 102, such that the pin 102 does not protrude through the mount 14, and the mount 14 defines or carries an alternative mounting axle for the device mount 22. This configuration of the patient mount 14 may be useful where the device mount 22 would benefit from being positioned on a mounting axle having a larger diameter than that defined by the pin 102, and/or where a bearing surface between device mount 22 and patient mount 14 is required, for example to reduce rotational friction. In such scenarios, the mounting axle of the patient mount 14 may be formed from a self-lubricating material, such as nylon, or carry a thrust bearing mechanism.

In further embodiments (not illustrated), the patient mount 14 is configured to receive the pin 102 therethrough and also define a sleeve portion extending partway along the pin 102 from the top portion 18 towards the protruding end 106 of the pin 102. In such embodiments, the pin 102 and the mount 14 together form a mounting axle for carrying the device mount 22. In this configuration, the device mount 22 may receive part of the patient mount 14 and the pin 102 to be mounted on both components.

In yet other embodiments (not illustrated), the patient mount 14 is configured to be secured across and receive the end 106 of the pin 102, such that the pin 102 does not protrude through the mount 14, and the mount 14 defines a recess or aperture, such as within a tubular sleeve structure. In such embodiments, the device mount 22 defines or carries a mounting axle protruding from the bottom portion 28 and dimensioned to fit within the sleeve structure to allow positioning the bottom portion 24 to be immediately adjacent and spaced from the top portion 18 of the patient mount 14.

In the illustrated embodiment 100, the device mount 22 is secured across a base portion of the device 12. The device 12 may be configured as a surgical navigation device, such as including one or more sensors operable to allow determining orientation of the device 12, and the device mount 22 be releasably or permanently secured to the device 12 to form a base of a housing of the device 12. Alternatively, the device 12 may be configured as a tracker configured for optical image tracking of the device 12, or configured for radiographic image tracking of the device 12, and the device mount 22 be releasably or permanently secured to the tracker, or integrally formed with the tracker. It will be appreciated that the above mentioned devices 12 are exemplary and that the device mount 22 is configurable to be secured to a wide range of other surgical devices.

Best shown in FIG. 3, the device mount 22 is configured such that the recess 28 receives the defined length of the pin 102 so that the end 106 of the pin 102 presses against an end wall 32 of the recess 28. In this embodiment 100, the end wall 32 covers across the recess 28. It will be appreciated that the end wall is alternatively configurable to be defined by shoulder or step in the recess 28.

Mounting the pin 102 into the recess 28 inhibits axial movement of the device mount 22 relative to the pin 102 but allows the device mount 22 to rotate freely about the axis of the pin 102. When the pin 102 is received in the recess 28 to contact the end wall 32, this arranges the bottom portion 24 of the device mount 22 to be immediately adjacent and spaced from the top portion 18 of the patient mount 14. As shown in FIG. 3, this defines a gap between the mounts 14, 22. The mounts 14, 22 are typically configured so that the gap is small, in the region of 1 mm or less, so that the mounts, 14, 22, in use, are close together but not in contact. This allows the magnetic bodies 20, 26 to attract each other sufficiently firmly to inhibit relative rotation of the mounts 14, 22, while minimising rotational friction between the device mount 22 and the pin 102 to allow a user to manually rotate the device mount 22 relative to the patient mount 12 to allow separating the mounts 14, 22 and without causing the pin 102 to be unscrewed from the patient.

In the illustrated embodiment, the recess 28 is dimensioned to have a clearance between the pin 102 and sidewall of the recess 28 such that only the end wall 32 is in contact with the pin 102. In other embodiments, the recess 28 is dimensioned to slidingly engage the pin 102. In further embodiments, the recess 28 is dimensioned to frictionally engage the pin 102.

In this embodiment 100, a pair of the first magnetic bodies 20 are carried by the patient mount 14, and a pair of the second magnetic bodies 26 are carried by the device mount 22. The bodies 20, 26 are each spaced by the respective mount 14, 22 in a complementary annular array to be spaced evenly about, and radially away from, the pin 102 or recess 28. In this embodiment 100, each of the magnetic bodies 20, 26 comprises a permanent magnet. In other embodiments, only one of the first magnetic bodies 20 and the second magnetic bodies 26 comprise permanent magnets, and the other of the bodies 20, 26 are at least partially formed from a magnetisable material, such as comprising a ferrous metal or a ferrite. A magnetic field may be induced into such a material to allow causing the bodies 20, 26 to act as temporary magnets.

The first magnetic bodies 20 are arranged by the patient mount 14 to have corresponding polarity to each other, and the second magnetic bodies 26 are arranged by the device mount 22 to have corresponding polarity to each other, and opposite polarity to the first magnetic bodies 20. Best shown in FIG. 3, the patient mount 14 arranges the first bodies 20 to be parallel to each other and in the same orientation, so that the magnetic vectors of the first bodies 20 are in the same direction, in this embodiment being the South end of each first body 20 arranged towards the top portion 18 of the mount 14. The device mount 22 arranges the second bodies 26 to be parallel to each other and in the same orientation, so that the magnetic vectors of the second bodies 26 are in the same direction, in this embodiment being the North end of each second body 26 arranged towards the bottom portion 24 of the mount 22. Arranging the magnetic bodies 20, 26 in this way means that the magnetic fields of the bodies 20, 26 attract the bodies 20, 26 towards each other when the device mount 22 is mounted on the pin 102 adjacent the patient mount 14 and the magnetic bodies 20, 26 are aligned. The position of the bodies 20, 26 in evenly spaced pairs about the pin 102 or recess 28, and corresponding polarity of the bodies 20, 26 of each pair, means that the device mount 22 may be retained relative to the patient mount 14 by magnetic force to inhibit relative rotation at one of two relative positions spaced 180 degrees apart from each other about the axis of the pin 102.

It will be appreciated that the mounts 14, 22 are configurable to carry more, or less, magnetic bodies 20, 26 than the pairs shown in the illustrated embodiment 100, including only a single first magnetic body 20 and a single second magnetic body 26, and/or that the orientation of the magnetic bodies 20, 26 is alternatively configurable to affect the magnetic vector direction, which can affect the relative mounting positions of the device mount 22 and patient mount 14. It will also be appreciated that one mount 14, 22 may carry more magnetic bodies 20, 26 than the other mount 14, 20, and/or that the bodies 20, 26 may be spaced unevenly about the pin 102 or recess 28.

For example, in some embodiments (not illustrated), each mount 14, 22 arranges each body 20, 26 of each pair of magnetic bodies 20, 26 to have alternative polarity, e.g. North-South, meaning that the mounts 14, 22 are only drawn towards each other when the bodies 20, 26 are aligned at one relative position of the mounts 14, 22. Alternatively, in other embodiments (not illustrated), each mount 14, 22 carries a set of three first or second magnetic bodies 20, 26 arranged in an annular array to have partially corresponding polarity, i.e. North-North-South, meaning that the mounts 14, 22 are only drawn towards each other when the bodies 20, 26 are aligned at one relative position of the mounts 14, 22. In yet other embodiments (not illustrated), each mount 14, 22 carries the set of three first or second magnetic bodies 20, 26 arranged in an annular array to have corresponding polarity, e.g. North-North-North, meaning that the mounts 14, 22 are drawn towards each other when the bodies 20, 26 are aligned at any of three relative positions of the mounts 14, 22. In further embodiments (not illustrated), each mount 14, 22 carries a set of four first or second magnetic bodies 20, 26 arranged in an annular array to have corresponding polarity, e.g. North-North-North-North, meaning that the mounts 14, 22 are drawn towards each other when the bodies 20, 26 are aligned at any of four relative positions of the mounts 14, 22.

In the illustrated embodiment 100, when the second magnetic bodies 26 are placed in proximity to the first magnetic bodies 20, this draws the device mount 22 towards the patient mount 14 to be arranged in the closest of the two defined relative positions of the mounts 14, 22, such as at 0° or 180° about the axis of the pin 102. In some embodiments, the patient mount 14 may carry one or more further magnets (not shown) interposed equally between the first magnetic bodies 20 and arranged to have non-corresponding polarity to the first magnetic bodies 20, typically having corresponding polarity to the second magnetic bodies 26. In such embodiments, rotating the device mount 22 a quarter turn away from one of the two defined relative positions, such as to 90° about the axis, aligns the second magnetic bodies 26 and further magnetic bodies to repel the device mount 22 away from the patient mount 14, effectively causing the device mount 22 to lift, before the mounts 14, 22 are drawn together again in the other of the two defined relative positions. This arrangement can inhibit the mounts 14, 22 being arranged relative to each other between the two defined relative positions, and consequently aid forcing the mounts 14, 22 towards one of the two positions.

FIG. 4A illustrates the device 12, joined to the device mount 22, mounted on the patient mount 14 and being rotated towards one of the two relative mounting positions. FIG. 4B illustrates the device 12 and device mount 22 arranged at one of the two mounting positions such that the second magnetic bodies 26 are aligned with the first magnetic bodies 20. In this position, the magnetic fields formed by the bodies 20, 26 draws the bodies 20, 26 towards each other to retain the mounts 14, 22 in the relative mounting position.

In the illustrated embodiment 100, the patient mount 14 carries a third magnetic body 34, configured as a permanent magnet, arranged to allow indexing the relative position of the patient mount 14 and the device mount 22. The third body 34 is arranged between the pair of first bodies 20 so that, viewed from above the top portion 18, the patient mount 14 is asymmetrical about the axis of the pin 102. The third body 34 is typically orientated such that its magnetic vector is non-corresponding to the magnetic vectors of the first bodies 20. In this embodiment 100, the third body 34 is arranged to have its magnetic vector parallel and opposed to the magnetic vectors of the first bodies 20. This can usefully repel the second magnetic bodies 26 of the device mount 22 to encourage rotation towards one of the two defined mounting positions. In other embodiments (not illustrated), the third body 34 may be arranged such that its magnetic vector is transverse to the magnetic vector of the first bodies 20. In yet other embodiments, the third body 34 is arranged such that its magnetic vector is corresponding to the magnetic vectors of the first bodies 20. In such embodiments, the third body 34 is typically configured to have a weaker magnetic field than the first bodies 20 to avoid the second bodies 26 being drawn and retained to the third body 34. In any of the embodiments, the arrangement of the third body 34 spaced from the first bodies 20 allows a sensor operable to detect magnetic field and carried by the device mount 22, or by the device 12 itself, to determine proximity of the third body 34 and, as a result, determine orientation of the device 12 (carrying the device mount 22) relative to the patient mount 14.

FIG. 4C illustrates the device 12 joined to the device mount 22 and mounted on the patient mount 14 to be at one of the two relative mounting positions such that the first and second magnetic bodies 20, 26 are aligned (not shown in this figure for simplicity). The device 12 houses two PCBs 36, only one of which carrying the sensor configured to detect magnetic field. Responsive to the sensor detecting the magnetic field of the third magnetic body 24, the device 12 may determine whether the device 12 is mounted to the patient mount 14, and may determine its orientation relative to the patient mount 14.

In the drawings, reference numeral 50 generally designates an assembly 50 for releasably mounting a surgical device 12 to a surgical tool having a shaft (not illustrated), such as an acetabular cup impactor, or the like. The assembly 50 includes: a tool mount 52 having one side defining a receiving portion 54 for receiving part of the shaft, and an opposed side defining an engagement portion 56, the receiving portion 54 associated with a retention mechanism 58 including a strap 60 securable across the receiving portion 54, the retention mechanism 58 operable to adjust an effective length of the strap 60 to trap the shaft between the strap 60 and the receiving portion 54; and a device mount 62 fixedly securable to the surgical device 12, the device mount 62 configured to releasably engage the engagement portion 56 of the tool mount 52 to allow mounting the surgical device 12 to the surgical tool.

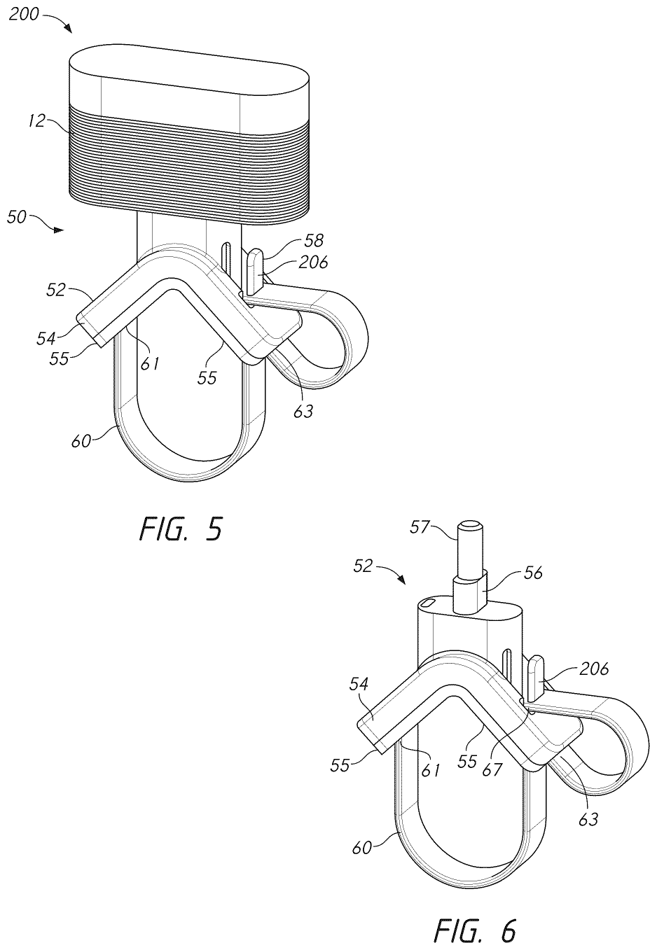

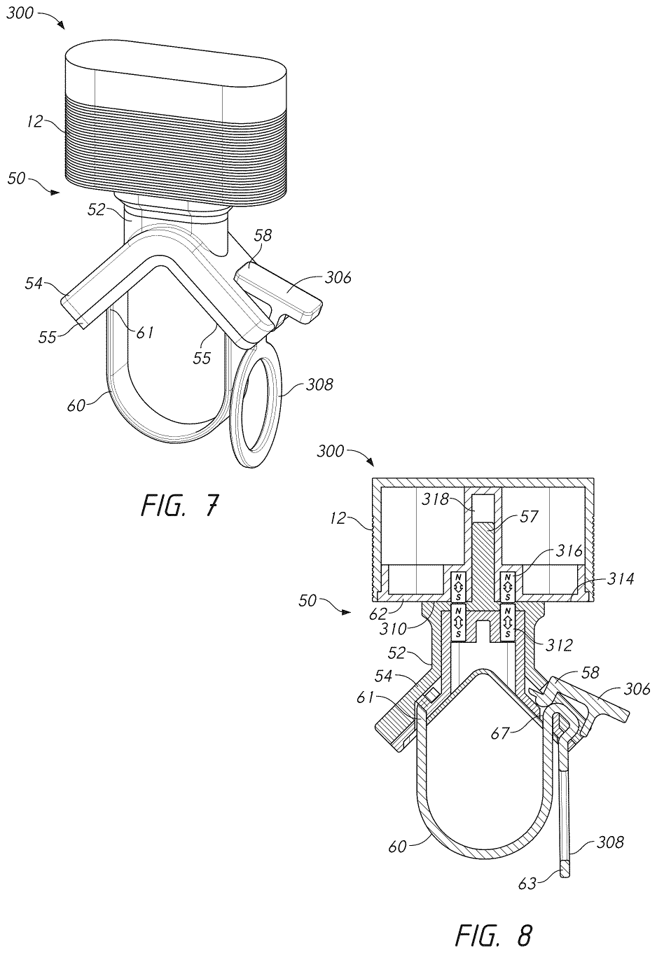

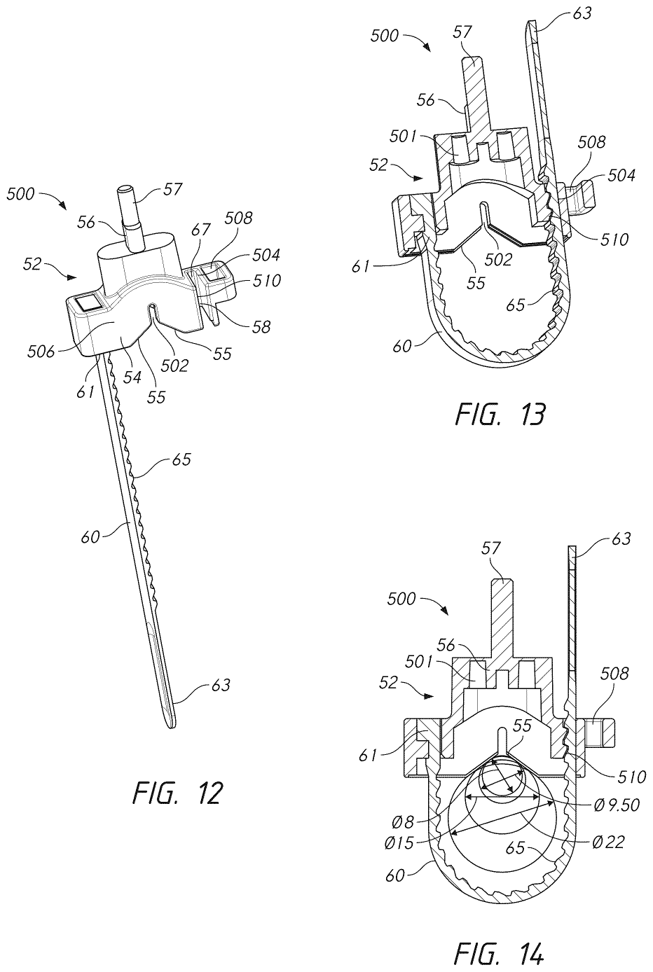

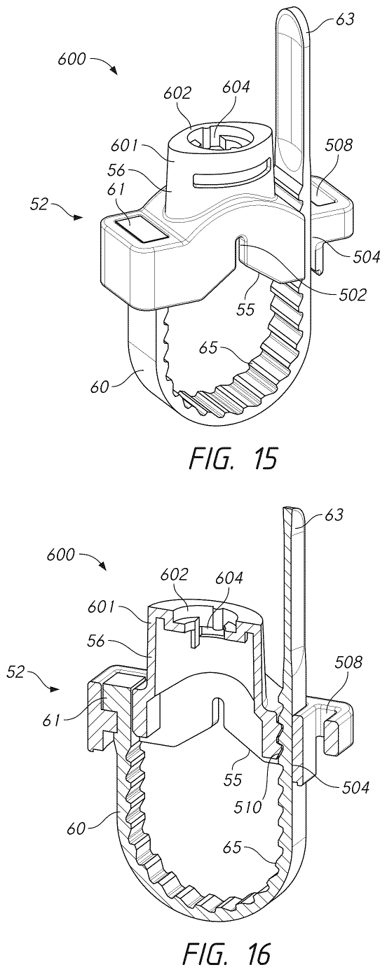

FIGS. 5 to 8 and 12 to 16 show embodiments of the assembly 50. FIGS. 5 and 6 show a first embodiment 200 of the assembly 50. FIGS. 7 and 8 shows a second embodiment 300 of the assembly 50. FIGS. 12 to 14 show a third embodiment 500 of the tool mount 52 of the assembly 50. FIGS. 15 and 16 shows a fourth embodiment 600 of the tool mount 52 of the assembly 50. It will be appreciated that common reference numerals identify common features unless indicated otherwise.

Each of the illustrated embodiments of the tool mount 52 have a receiving portion 54 including a pair of abutment surfaces 55. The surfaces 55 are arranged to diverge away from each other to allow receiving the shaft between the surfaces 55. In the illustrated embodiments, the surfaces 55 are joined, or are adjacent each other, to form a V-shaped structure for receiving the tool shaft and firmly supporting the tool mount 52 on the shaft. It will be appreciated that, in other embodiments (not illustrated), the surfaces 55, may be separate and spaced from each other, such as defined by spaced pads arranged to abut the tool. In yet other embodiments (not illustrated), the angle of the abutment surfaces 55 relative to each other is adjustable, such as by each surface being defined by a body pivotally connected to a central chassis. In further embodiments (not illustrated), the receiving portion 54 includes only a single abutment surface 55 arranged to abut the tool shaft. In such embodiments, the single abutment surface may be formed from, or carry, a substantially resiliently deformable material to allow partially wrapping around the tool shaft to enhance grip.

Each of the abutment surfaces 55 are shaped to be planar to allow fitting to a range of different tool shaft diameters, such as the 8 mm, 9.5 mm, 15 mm, and 22 mm shaft diameters illustrated in FIG. 14. It will be appreciated that, in other embodiments, one or both of the abutment surfaces 55 may be at least partially curved. Typically, each surface 55 is formed from, or carries, a resiliently deformable material, such as an elastomer, to enhance the surfaces 55 gripping the shaft of the tool.

In the illustrated embodiments of the tool mount 52, the strap 60 is an elongate flexible member which is at least partially resiliently deformable, such as to allow stretching to an extent around the shaft of the instrument. The strap 60 may define structures to enhance gripping the shaft and/or being gripped by the retention mechanism 58, such as ridges 65 shown in FIGS. 12 to 16 and discussed further below. In some embodiments, one or both sides of the strap 60 defines any of grooves, teeth, dimples, or domes to affect gripping and/or being gripped. In some embodiments, the strap 60 is configured as a ratchet strap engageable by a ratchet mechanism of the retention mechanism 58.

In the illustrated embodiments 200, 300, 500, the engagement portion 56 of the tool mount 52 includes a protrusion in the form of a boss defining a cylindrical shaft 57 which is receivable by the device mount 62 to allow the mount 62 to be engaged with, and be rotatable relative to, the tool mount 52. In other embodiments (not illustrated), the boss defines a non-cylindrical shaft, such as a square or elliptical section shaft, to inhibit relative rotation of the mounts 52, 62. In yet other embodiments, the engagement portion 56 defines a thread or bayonet-type structure, such as the axially extending recess 602 illustrated in FIGS. 15 and 16 and discussed below, to allow axially and rotatably locking the device mount 62 to the tool mount 52. In embodiments 600 where the engagement portion 56 defines the recess 602, the device mount 62 (not shown in FIGS. 15 and 16) includes a connector portion, such as a post or other protrusion, dimensioned to slidingly engage the recess to allow mounting the device mount 62 to the tool mount 52.

Each illustrated embodiment of the tool mount 52 has one end 61 of the strap 60 fixedly secured towards one side of the receiving portion 54 and the other end 63 of the strap 60 passed through an aperture 67 dimensioned to receive the strap 60 and located towards the other side of the receiving portion 54 to allow the strap 60 to form a closed loop. In some embodiments, the first end 61 of the strap 60 is configured to be releasably securable relative to the receiving portion 54. This can allow releasing the first end 61 to fit the strap 60 around the surgical tool. Generally, the first end 61 is permanently secured at the side of the receiving portion 54 to prevent removal of the strap 60 and fitting to the tool involves operating the retention mechanism 58 to increase the effective length of the strap 60, thereby increasing the diameter of the loop, to allow fitting around the tool shaft.

The retention mechanism 58 of each embodiment 200, 300, 500, 600 is configured to retain the strap 60 in position when arranged to extend through the aperture 67.

The first embodiment 200 has a retention mechanism 58 including a depressible tab 206 associated with the aperture 67 and biased towards one side of the aperture 67. The strap 60 is arranged to extend through the aperture 67 such that the strap 60 is trapped by the tab 206 to inhibit the strap 60 being drawn through the aperture 67 in at least one direction. This arrangement means that the tab 206 must be manually depressed to allow increasing the effective length of the strap 60, such as to remove the tool mount 52 from the tool.

The second embodiment 300 has a retention mechanism 58 including a pivotable lever 306 associated with the aperture 67 and arranged to pivot across the aperture 67. The strap 60 is arranged to extend through the aperture 67 and the lever 306 is pivotable to trap the strap 60 and inhibit the strap 60 being drawn through the aperture 67 in at least one direction. This arrangement means that the lever 306 must be manually pivoted to allow increasing the effective length of the strap 60, such as to remove the tool mount 52 from the tool. Typically, the lever 306 is arranged such that pulling the second end 63 of the strap 60 away from the receiving portion 58, in the figures being in an upwards direction, causes the lever 306 to pivot and allow the strap 60 to be drawn through the aperture 67.

It will be appreciated that the retention mechanism 58 is configurable to include other features suitable to retain the strap 60 in position to secure the closed loop across the receiving portion 54. For example, in some embodiments (not illustrated), the retention mechanism 58 includes hook and loop fasteners fixed relative to the receiving portion 54, and carried by the strap 60. In such embodiments, the strap 60 is arrangeable such that the hook and loop fasteners engage to retain the strap in position. In other embodiments, the retention mechanism 48 includes a clasp or buckle-type mechanism, such as a side-release buckle, or a sliding magnetic buckle, fixed relative to the receiving portion 54 and arranged to receive and retain the strap 60.

The second end 63 of the strap 60 of the first embodiment 200 is joined to a side of the receiving portion 54. This arrangement fixes the strap 60 at both ends 61, 63 to prevent the strap 60 from being removed from the tool mount 52. It will be appreciated that the strap 60 of the second embodiment 300 may alternatively be configured in this way.

The second end 63 of the strap 60 of the second embodiment 300 includes a ring pull structure 308. This can enhance ease of gripping the strap 60 by the user. It will be appreciated that the strap 60 of the first embodiment 200 may alternatively be configured in this way.

As shown in FIG. 8, the engagement portion 56 of the illustrated embodiment 300 has an operatively top portion 310 defining the protrusion 57 and carrying a pair of first magnetic bodies 312 spaced apart from each other. The device mount 62 defines an operatively bottom portion 314 carrying a complementary pair of second magnetic bodies 316 spaced apart from each other, and defines a recess 318 dimensioned to receive at least a portion of the protrusion 57. At least one of the first magnetic bodies 312 and the second magnetic bodies 316 comprise magnets. Mounting the device mount 62 on the protrusion 57 and aligning the first magnetic bodies 312 with the second magnetic bodies 316 allows the magnets to draw the mounts 52, 62 towards each other. It will be appreciated that the pair of first magnetic bodies 312 and the pair of second magnetic bodies 316 are exemplary, and that the mounts 52, 62 may each carry more, or less, magnetic bodies 312, 316, including only a single magnetic body 312, 316.

In this embodiment 300, the protrusion 57 is configured to allow relative rotation of the mounts 52, 62. When the first and second magnetic bodies 312, 316 are aligned, the magnetic force of the bodies 312, 316 is typically configured to inhibit relative rotation to position the bodies in one, or more, defined relative positions.

It will be appreciated that the first embodiment 200 is configurable in the same way such that magnetic bodies cause retaining the mounts 52, 62 together. It will also be appreciated that the mounts 52, 62 of either embodiment 200, 330 are alternatively configurable to have more, or less, magnetic bodies 312, 316, and/or the bodies 312, 316 are arrangeable in different ways, such as to alternatively direct the magnetic vectors, to affect retaining the mounts 52, 62 together in one or more relative positions, as described above with reference to the assembly 10.

In the illustrated embodiments, the device mount 62 is secured across a base portion of the device 12. The device 12 may be configured as a surgical navigation device, such as including one or more sensors operable to allow determining orientation of the device 12, and the device mount 62 be releasably or permanently secured to the device 12 to form a base of a housing of the device 12. Alternatively, the device 12 may be configured as a tracker configured for optical image tracking of the device 12, or configured for radiographic image tracking of the device 12, and the device mount 62 be releasably or permanently secured to the tracker, or integrally formed with the tracker. It will be appreciated that the above mentioned devices 12 are exemplary and that the device mount 62 is configurable to be secured to a wide range of other surgical devices.

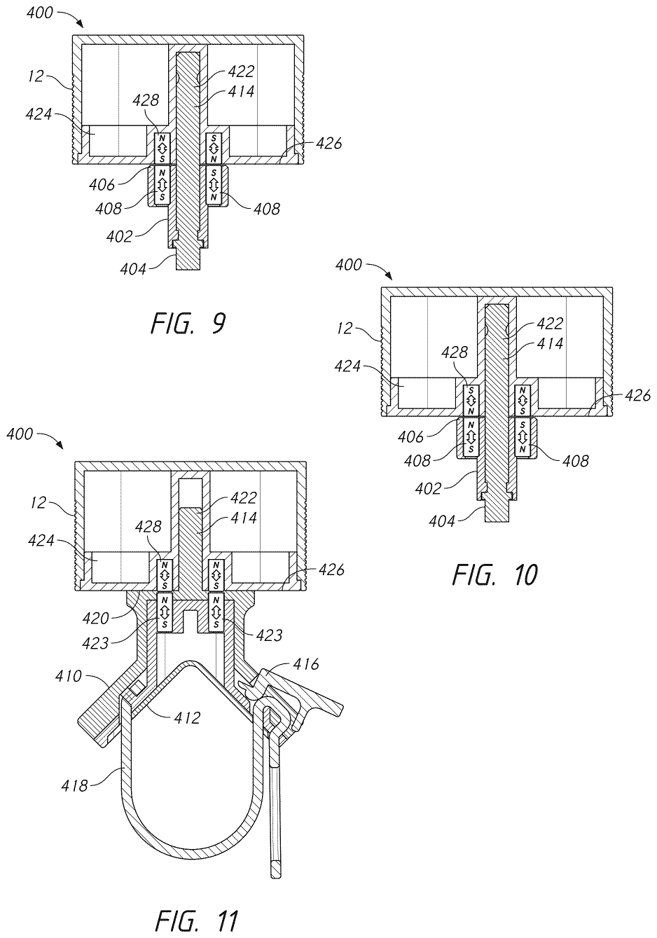

FIGS. 9 to 11 show a kit 400 for releasably mounting a surgical device 12 to a patient (not illustrated) or a surgical tool having a shaft (not illustrated). The kit 400 includes:

a patient mount 402 fixedly securable to a pin 404, the pin 404 configured for threadedly engaging the patient, the patient mount 402 defining an operatively top portion 406 carrying at least one first magnetic body 408, in this embodiment 400 carrying a pair of the first magnetic bodies 408 spaced apart from each other;

a tool mount 410 having a receiving portion 412 for receiving part of the shaft, and an opposed engagement portion 414, the receiving portion 412 associated with a retention mechanism 416 including a strap 418 securable across the receiving portion 412, the retention mechanism 416 operable to adjust an effective length of the strap 418 to trap the shaft between the strap 418 and the receiving portion 412, the engagement portion 414 having an operatively top portion 420 defining a protrusion 422, and the top portion 420 carrying at least one second magnetic body 423, in this embodiment 400 carrying a pair of the second magnetic bodies 423 spaced apart from each other; and

a device mount 424 fixedly securable to the surgical device 12, the device mount 424 defining an operatively bottom portion 426 carrying at least one third magnetic body 428, in this embodiment 400 carrying a pair of the third magnetic bodies 428 spaced apart from each other, the bottom portion 426 configured to receive part of at least one of the pin 404 and the patient mount 402, and the bottom portion 426 further configured to at least partially receive the protrusion 422.

At least two of the first, second and third magnetic bodies 408, 423, 428 comprise magnets. Mounting the device mount 424 on at least one of the pin 404 and the patient mount 402, or on the protrusion 422, and aligning the third magnetic bodies 428 with the first magnetic bodies 408, or aligning the third magnetic bodies 428 with the second magnetic bodies 423, allows the magnets to draw the mounts 424, 402, 410 towards each other to retain the surgical device 12 to the patient or the surgical tool.

In the illustrated embodiment 400, the patient mount 402 carries a pair of the first magnetic bodies 408 spaced evenly apart and radially away from the pin 404, each body 408 comprising a permanent magnet. The magnets are arranged to have opposing polarity, where one body 408 is arranged to have its North end adjacent the top portion 406 and the other body 408 is arranged to have its South end adjacent the top portion 406.

The tool mount 410 carries a pair of the second magnetic bodies 423, each comprising permanent magnets. The magnets are arranged to have corresponding polarity, where one body 423 is arranged to have its North end adjacent the top portion 420 and the other body 423 is arranged to have its North end adjacent the top portion 420.

The device mount 424 carries a pair of the third magnetic bodies 428, each comprising magnetisable material, such as a ferrous metal or a ferrite. A magnetic field may be induced to the third magnetic bodies 428 to allow acting as temporary magnets. Each third body 428 is associated with a sensor (not shown) operable to determine magnetic polarity, such as a Hall effect sensor. The sensor may be mounted to the device mount 424 or the device 12.

FIG. 9 shows the device mount 424 mounted to the patient mount 402 in a first orientation such that the third magnetic bodies 428 are aligned with the first magnetic bodies 408. When arranged in this way, the magnetic fields of the first bodies 408 cause the third bodies 428 to adopt opposing polarity. This causes the sensor to detect a first reading, in this scenario being North-South, to allow determining the device mount 424 is arranged on the patient mount 402 in the first relative position.

FIG. 10 shows the device mount 424 mounted to the patient mount 402 in a second orientation such that the third magnetic bodies 428 are aligned with the first magnetic bodies 408. When arranged in this way, the magnetic fields of the first bodies 408 cause the third bodies 428 to adopt opposing polarity. This causes the sensor to detect a second reading, in this scenario being South-North, to allow determining the device mount 424 is arranged on the patient mount 402 in the second relative position.

FIG. 11 shows the device mount 424 mounted to the tool mount 410 such that the third magnetic bodies 428 are aligned with the second magnetic bodies 423. When arranged in this way, the magnetic fields of the second bodies 423 cause the third bodies 428 to adopt opposing polarity. This causes the sensor to detect a third reading, in this scenario being North-North, to allow determining the device mount 424 is arranged on the tool mount 410.

It will be appreciated that the arrangement of the first magnetic bodies 408 and second magnetic bodies 423 is exemplary, and that the bodies 408, 423 may be alternatively arranged to allow the sensor to determine polarity and, as a result, determine which of the mounts 402, 410 the device mount 424 is mounted on, and in which relative position.

FIGS. 12 to 14 show the third embodiment 500 of the tool mount 52. This embodiment 500 is engageable with the device mount 62 shown in FIGS. 5, 7 and 8, and may carry the magnetic members 312 (not shown in these figures) in recesses 501. As discussed above, the third embodiment 500 shares features with the previously described embodiments, and common reference numerals indicate common features.

The receiving portion 54 of this embodiment 500 includes the abutment surfaces 55 adjacent to each other and separated by a gap 502. As shown in FIG. 14, the surfaces 55 are arranged to diverge away from each other to allow receiving a wide range of shaft diameters, with common surgical tool diameters illustrated in the figure.

The strap 60 is arrangeable across the receiving portion 54 to be received in the aperture 67. In this embodiment 500, the aperture 67 is configured as a channel 504 which opens out on one side 506 of the mount 52. This configuration of the aperture 67 allows the strap 60 to be slid into and out of the open side of the channel 504, which can enhance ease of securing the strap 60 about the instrument shaft when placed against the abutment surfaces 55, or removing the strap 60 from the channel 504 and consequently releasing the instrument shaft. A second aperture 508 is defined adjacent the channel 504 and dimensioned to receive the strap 60 to allow folding the strap 60 back on itself. This can arrange the strap 60 to form a tortuous path to enhance securely fitting the strap 60 across the receiving portion 54 and/or conveniently house the free end 63 of the strap 60, for example, to inhibit the end 63 snagging on other objects during use.

The retention mechanism 58 in this embodiment 500 includes a plurality of teeth 510 arranged in the channel 504 and shaped to engage the ridges 65 of the strap 60. The open-sided configuration of the channel 504 means that sliding the strap 60 into the channel 504 can slidingly engage the strap 60 with the teeth 510. Best shown in FIGS. 13 and 14, the teeth 510 and ridges 65 may be complementarily shaped to provide a ratchet-type of function, where the teeth 510 and ridges 65 engage to inhibit the strap 60 travelling through the channel 504 in one direction, which would loosen the strap 60 from the shaft, while allowing the strap to travel through the channel 504 in the opposite direction, which would tighten the strap 60 about the shaft. It will be appreciated that, in other embodiments (not shown), the teeth 510 may be substituted with a ratchet mechanism operable to engage the strap 60, such as including a biased, depressible tab configured to urge the strap 60 against a sidewall of the channel 504.

FIGS. 15 and 16 show the fourth embodiment 600 of the tool mount 52. This embodiment 600 shares features with the previously described embodiment 500, and common reference numerals indicate common features. The embodiment 600 is configured to form a bayonet coupling with a complementary device mount 62 (not shown) to facilitate removably engaging the device mount 62 with the tool mount 52.

The engagement portion 56 of this embodiment 600 includes a sleeve section 601 defining the axially extending recess 602. The recess 602 defines a slot 604 shaped to receive a complementarily shaped connector portion of the device mount 62. Displacing the connector portion through the slot 604 and causing relative rotation of the device mount 62 and the tool mount 52 engages the mounts 62, 53 together. While the device mount 62 is configured to have a male portion of the bayonet coupling and the tool mount 52 is configured to have a female portion of the bayonet coupling in this embodiment 600, it will be appreciated that this configuration may be reversed. Furthermore, it will be appreciated that each mount 52, 62 may be alternatively shaped and dimensioned to provide a portion of a bayonet coupling, and that a wide range of structures are within the scope of this disclosure.

Use of the assembly 10 may involve: securing the patient mount 14 to the pin 16; securing the device mount 22 to the device 12; engaging the pin 16 with the patient; placing the device mount 22 on to the pin 16 such that the pin 16 is received into the recess 28 to arrange the bottom portion 24 of the device mount 22 immediately adjacent and spaced from the top portion 18 of the patient mount 14; and aligning the first magnetic bodies 20 with the second magnetic bodies 26 to allow the magnets to draw the mounts 14, 22 towards each other to inhibit relative rotation of the mounts 14, 22.

Use may also involve repositioning the device mount 22 on the patient mount 14 by rotating the device 12 or device mount 22 with sufficient torque to overcome the magnetic attraction between the magnetic bodies 20, 26, then realigning the magnetic bodies 20, 26 with the device mount 22 rotated around 180 degrees from its initial position relative to the patient mount 14 to allow the magnets to draw the mounts 14, 22 towards each other to inhibit relative rotation of the mounts 14, 22.

Use may also involve removing the device mount 22 from the patient mount 14 by rotating the device 12 or device mount 22 with sufficient torque to overcome the magnetic attraction between the magnetic bodies 20, 26, then displacing the device mount 22 axially along the pin 16 and away from the patient mount 14 to withdraw the pin 16 from the recess 28.

Use of the assembly 50 may involve: arranging the tool mount 52 about the shaft of the tool, typically by operating the retention mechanism 58 and adjusting the effective length of the strap 60 to allow spaced to pass the shaft, and for some tools also a handle, between the strap 60 and the receiving portion 54; securing the tool mount 52 to the shaft, typically by adjusting the effective length of the strap 60 to trap the shaft between the strap 60 and the receiving portion 54; securing the device mount 62 to the device 12; and engaging the device mount 62 with the engaging portion 56 of the tool mount 52.

The assembly 10 releasably mounts the device 12 to a single pin 16 threadedly engaged with the patient. This can be useful as this minimises patient discomfort and/or injury, and/or enhances efficiency of surgery, by requiring only a single pin to fixedly mount the device 12 to the patient. The assembly 10 may also advantageously allow the device to be readily removed from, and repositioned relative to, the pin 16 while minimising friction between the mounts 14, 22, and/or minimising friction between the device mount 22 and the pin 16, which can inhibit transferring torque to the pin 16, which could cause the pin to be disengaged from the patient.

The assembly 50 releasably mounts the device 12 to a shaft of a tool. The arrangement of the retractable strap 60 and receiving portion 54 can allow the tool mount 52 to be readily mounted on a range of shaft shapes and/or diameters. Furthermore, the tool mount 52 may provide a secure mounting mechanism which can be readily fitted to, and removed from, the tool, which can enhance efficiency of surgery.

The kit 400 releasably mounts the device 12 to a single pin 16 threadedly engaged with the patient, or releasably mounts the device 12 to a shaft of a tool. This can usefully enhance ease of transferring the device 12 from being mounted to the patient to being mounted to the tool, and vice versa, which can enhance efficiency of surgery.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. An assembly for releasably mounting a surgical device to a patient, the assembly including:
a patient mount fixedly securable to a pin, the pin configured for threadedly engaging the patient, the patient mount defining an operatively top portion carrying at least one first magnetic body; and
a device mount fixedly securable to the surgical device, the device mount defining an operatively bottom portion carrying a complementary at least one second magnetic body, the bottom portion defining a recess dimensioned to receive a defined length of at least one of the pin and the patient mount and allow rotating the device mount relative to the patient mount,
one or more of the at least one first magnetic body and the at least one second magnetic body comprises a magnet, and whereby mounting the recess of the device mount on at least one of the pin and the patient mount arranges the bottom portion of the device mount immediately adjacent and spaced from the top portion of the patient mount, and aligning the at least one first magnetic body with the at least one second magnetic body allows the one or more magnets to draw the mounts towards each other to inhibit relative rotation of the mounts.

2. The assembly of claim 1, wherein the patient mount carries a pair of the first magnetic bodies spaced apart from each other, each of the first magnetic bodies comprising magnets arranged to have corresponding polarity, and the device mount carries a pair of the second magnetic bodies spaced apart from each other, each of the second magnetic bodies comprising magnets arranged to have corresponding polarity to each other and opposite polarity to the first magnetic bodies.

3. The assembly of claim 2, wherein the top portion of the patient mount carries a third magnetic body interposed between the pair of first magnetic bodies, the third magnetic body comprising a magnet arranged to have non-corresponding polarity to the first magnetic bodies.

4. The assembly of claim 1, wherein the patient mount carries a pair of the first magnetic bodies spaced apart from each other, each of the first magnetic bodies comprising permanent magnets, and the device mount carries a pair of the second magnetic bodies spaced apart from each other, each of the second magnetic bodies comprising magnetisable material to allow forming temporary magnets.

5. The assembly of claim 2, wherein the patient mount is configured to arrange the pair of the first magnetic bodies to be evenly spaced radially away from the pin, and the device mount is configured to arrange the pair of the second magnetic bodies in complementary spacing to the first magnetic bodies, to allow mounting the device mount on the at least one of the pin and the patient mount and aligning the first magnetic bodies with the second magnetic bodies in one of two relative positions.

6. The assembly of claim 1, wherein the patient mount is securable along the pin such that an end of the pin is spaced from the top side by more than the defined length to allow mounting the device mount on the pin.

7. The assembly of claim 6 including the pin, and wherein the patient mount is permanently joined to the pin.

8. The assembly of claim 6 including the pin, wherein the patient mount is integrally formed with the pin.

9. An assembly for releasably mounting a surgical device to a patient, the assembly including:
a patient mount including a pin configured for threadedly engaging the patient, the patient mount defining an operatively top portion carrying at least one first magnetic body; and
a device mount fixedly securable to the surgical device, the device mount defining an operatively bottom portion carrying a complementary at least one second magnetic body, the bottom portion defining a recess dimensioned to receive the patient mount and allow rotating the device mount relative to the patient mount,
one or more of the at least one first magnetic body and the at least one second magnetic body comprises a magnet, and whereby mounting the recess of the device mount on the patient mount arranges the bottom portion of the device mount immediately adjacent and spaced from the top portion of the patient mount, and aligning the at least one first magnetic body with the at least one second magnetic body allows the one or more magnets to draw the mounts towards each other to inhibit relative rotation of the mounts.

10. A surgical device assembly including:
a surgical navigation device operable to determine orientation; and
the assembly of claim 9, wherein the device mount forms a base portion of the surgical navigation device.

11. The surgical device assembly of claim 10, wherein the surgical navigation device includes one or more sensors operable to detect magnetic field, and the surgical navigation device is configured to determine when the device is mounted to the patient mount responsive to the one or more sensors detecting the magnetic field of one or more of the magnetic bodies.

12. The surgical device assembly of claim 11, wherein the surgical navigation device is configured to determine orientation of the device relative to the patient mount responsive to the one or more sensors detecting the magnetic field of one or more of the magnetic bodies.

13. An assembly for releasably mounting a surgical device to a surgical tool having a shaft, the assembly including:
a tool mount having one side defining a receiving portion for receiving part of the shaft, and an opposed side defining an engagement portion, the receiving portion associated with a retention mechanism including a strap securable across the receiving portion, the retention mechanism operable to adjust an effective length of the strap to trap the shaft between the strap and the receiving portion; and
a device mount fixedly securable to the surgical device, the device mount configured to releasably engage the engagement portion to allow mounting the surgical device to the surgical tool.

14. The assembly of claim 13, wherein the receiving portion includes at least one pair of abutment surfaces, the surfaces of the, or each, pair arranged to diverge away from each other to allow receiving the part of the shaft between the surfaces.

15. The assembly of claim 14, wherein the receiving portion includes a pair of the abutment surfaces, each of the surfaces being planar.

16. The assembly of claim 13, wherein the tool mount defines an aperture dimensioned to receive the strap, and the retention mechanism is configured to retain the strap in position when arranged to extend through the aperture.

17. The assembly of claim 16, wherein the retention mechanism includes teeth disposed within the aperture, and the strap is configured to engage the teeth.

18. The assembly of claim 17, wherein the aperture defines a channel housing the teeth, and the strap defines projections shaped to slidingly engage the teeth as the strap is slid into the channel.

19. The assembly of claim 16, wherein the retention mechanism includes a depressible tab associated with the aperture, and whereby the tab is biased towards one side of the aperture to trap the strap and inhibit the strap being drawn through the aperture in at least one direction.

20. The assembly of claim 16, wherein the retention mechanism includes a pivotable member associated with the aperture, and whereby pivoting the pivotable member to a locking position traps the strap to inhibit the strap being drawn through the aperture in at least one direction.

21.-34. (canceled)
